(19) United States
(12) Patent Application Publication
(10) Pub. No.: US 2026/0287533 A1
(43) Pub. Date: Sep. 24, 2026

(54) SENSOR ELEMENT WITH MEMBRANE AND COMBINED HEATING AND TEMPERATURE-MEASURING CONDUCTOR PATHS

(71) Applicant: ARCHIGAS GMBH, Ruesselsheim (DE)

(72) Inventors: Illya KAUFMAN, Frankfurt/Main (DE); Wladimir BARSKYI, Frankfurt/Main (DE); Friedemann VOELKLEIN, Niedernhausen (DE)

(21) Appl. No.: 19/475,754

(22) PCT Filed: Apr. 15, 2024
(86) PCT No.: PCT/EP2024/060141

§ 371 Date: Oct. 16, 2025

(30) Foreign Application Priority Data
Apr. 17, 2023 (DE) 10 2023 109 571.4

(51) Int. Cl. G01N 25/18; G01N 33/00; H05B 3/00; H05B 3/03

(52) U.S. Cl. CPC G01N 25/18; G01N 33/0027; H05B 3/0019; H05B 3/03

(57) ABSTRACT

A sensor element for measuring thermal properties of a fluid medium includes a membrane, a cover and a frame surrounding the membrane on at least two sides. The membrane is separated from the frame in regions by recesses, is self-supporting, and is supported by the frame via strips, with the sensor element having at least one electrical resistance element on the frame and an electrical heating element on the membrane. The electrical heating element is electrically contacted via connecting conductor paths running from the frame via at least one of the strips to the membrane. The frame has an electrical heating element on the frame, which surrounds the at least one electrical resistance element on the outside on at least two sides.

### RELATED APPLICATIONS

The present application is a National Phase of International Application Number PCT/EP2024/060141, filed Apr. 15, 2024, and claims priority of German Application Number 10 2023 109 571.4 filed Apr. 17, 2023.

### FIELD

The disclosure relates to a sensor element for measuring thermal properties of a fluid medium, for example, for measuring the thermal conductivity and/or the hydrogen gas content, the hydrocarbon gas content or the helium content of a fluid medium. The disclosure also relates to a sensor comprising such a sensor element and to a method for determining at least one thermal property of a fluid medium, for example, the thermal conductivity and/or the hydrogen content, the hydrocarbon gas content or the helium content of the fluid medium.

### BACKGROUND

The disclosure relates in at least some embodiments to a sensor element and to a method for determining the hydrogen concentration in a moist oxygen-gas mixture or a moist air-gas mixture using the thermal conductivity of the oxygen or air-gas mixture and to the application of the method and the usability of the sensor element in the case of fluctuating ratios of the individual air components as well as the moisture content of the air-gas mixture or oxygen-gas mixture. In at least some embodiments, the fluid medium is a gas, for example, an air-gas mixture.

Heat conduction measurements may be used in industrial gas analysis to determine the gas concentration in binary or quasi-binary gas mixtures. A binary mixture is a mixture of exactly two different gases in any proportions. If a measuring component consists of several gases that behave like a single gas with regard to their thermal conductivity, in a binary measurement of the gas mixtures it is referred to as a quasi-binary gas mixture. In at least some embodiments, this is the case when these components remain permanently in unchanged proportions, such as in dry atmospheric air, or when the components of this mixture have the same thermal conductivity.

Thermal conductivity describes a physical property that indicates how well a substance conducts heat. The thermal conductivity value depends on temperature, and for each gas there is a specific, experimentally determined indicator λ(T), which is expressed in watts per meter and Kelvin (W/(m K)).

The measurement method is based on the determination of the heat output Q. In at least some embodiments, a heat source transfers heat energy to a heat sink via the surrounding gas. The heat output and the measurement are described by the defined geometry and the thermal conductivity (a material-specific function of temperature) as in the following Formula 1:

$$Q = λ ( T ) \frac{A ( {T}_{q} - {T}_{s} )}{d} \qquad ( 1 )$$

wherein, in Formula 1,
A is the area of the heat source (defined suspended area that is electrically heated by the heating element),
d is a defined (and constant) distance between the heat source and the heat sink (the walls of the measuring chamber), and relates to the walls being heated to desired temperature,
T_{q} is the temperature of the heat source and
Tₛ is the temperature of the heat sink.

The electrical power required to maintain the heat source at a given temperature is proportional to the thermal conductivity of the measured gas. In at least some embodiments, this electrical power is used as a measurement signal.

Silicon-based micromechanical sensors or micro-electro-mechanical systems (MEMS) are used, inter alia, for the measurement. Improved accuracy and improved reproducibility of the geometric parameters A and d in Formula 1 are provided using these sensors or MEMS in at least some embodiments. In at least some embodiments, this provides improved reproducibility of the measurement results. The correlation of the thermal conductivity signal with the mixture of two gases results in the concentration measurement.

A disadvantage of the known sensors and sensor elements, whose functional principle is based on measuring the thermal conductivity of the surrounding gas, is that if another gas occurs in the binary gas mixture, an undefined signal is obtained that cannot be clearly associated with the concentration of the gases. In many applications where the hydrogen concentration needs to be determined, the gas mixtures are not purely binary. Another disadvantage of a measurement method based on MEMS technology is that the sensor may immediately suffer irreversible damage if it is contaminated with condensate.

More and more attention is paid to the safety monitoring of hydrogen concentration. In at least some embodiments, this includes monitoring the lower explosion limit or upper explosion limit (LEL or UEL). The hydrogen content in air or in oxygen or the air or oxygen content in hydrogen is measured. During the measurement, the air composition may change, for example, due to the enrichment or release of moisture in the measurement gas, e.g., due to an increase or decrease in the H₂O concentration in the measurement gas. Common applications for these measurement tasks are in the field of electrolysis or exhaust gas determination from fuel cells. In order to eliminate the influence of air moisture on the thermal conductivity measurement, currently either gas drying is carried out, which leads to an error in the measured sample, or an additional moisture measurement is carried out to compensate for the influence. Both are often used. In at least some embodiments, this helps to make setting up and servicing such sensors complicated and expensive.

EP 0 285 833 A2 discloses a method for determining the gas concentration in a gas mixture by measuring the thermal conductivity of the gas mixture. The thermal conductivity of the gas mixture is measured at as many gas temperatures as the number of gas components minus one. In at least some embodiments, this requires a large number of measurements at different temperatures. The individual gas concentrations are then calculated from the measured thermal conductivity values. To measure the thermal conductivities of the gas mixture at different gas temperatures, a sensor is used. In at least some embodiments, the sensor consists of a silicon carrier plate with etched thin-film resistors on which a cover plate rests. A channel is formed in the sensor through which the gas mixture is able to reach the measuring chamber. The sensor has an insulating layer through which heat is able to dissipate, which may negatively influence the measurement result because it obscures the actual measurement variable. In addition, thermal stresses may occur in the insulating layer, presenting challenges at high temperatures.

DE 10 2015 222 072 A1 discloses a heating device for a MEMS sensor in which a thin-film heating element with a special geometry is used to form a substantially constant electrical current density.

DE 10 2015 222 072 A1 is based on a prior art in which a membrane is applied to a chip or carrier, with the chip material being removed in some regions. As a result, the membrane is coupled to the chip, which may lead to inhomogeneous heat dissipation and temperature distribution on the membrane, which helps to make the special geometry of the thin-film heating element necessary for a more homogeneous distribution of the temperature on the membrane. Temperature measurement is not discussed.

DE 10 2016 200 267 A1 discloses a device for detecting a gas in which two heating elements are arranged on membranes and the measurement is carried out based on a comparison. In at least some embodiments, this design does not provide protection against condensation or a solution for cross-sensitivity in high moisture conditions. In addition, the membranes arranged one above the other make it difficult to hermetically seal the sensor to protect it against aggressive environments.

From US 2015/0097260 A1, a heat conduction sensor on a silicon wafer is known, which has a heat transfer recess with a flat bottom. A special feature of this sensor is that the heat transfer cavity is designed in such a way that no natural convection takes place inside and the heat transfer occurs exclusively through the thermal conductivity of the gas in the cavity. However, the structure leads to undefined heat transfer above the membrane. In at least some embodiments, this may lead to convection above the sensor and heat transfer to the compensation resistor without a cover may cause a position-dependent measurement. In addition, this design does not offer any protection against condensation or any reduction in cross-sensitivity to moisture.

DE 101 44 873 A1 discloses a thermal conductivity sensor with a porous cover. The construction provides basic protection against external influences and condensation. However, at high moisture or condensate levels, wetting of the porous structure may block gas access.

DE 10 2005 058 832 B4 discloses an improved heated H₂ sensor which helps reduce the influence of moisture by external heating of the measuring chamber and thus helps enable more precise measurements of hydrogen in gaseous media. Due to the external heating of the housing, a high electrical power is required. In addition, complete evaporation of condensate is not guaranteed, so that liquid could reach the membrane.

JP 2010-266265 A discloses an improved gas detector that increases the accuracy of detecting combustible gases by adjusting readings based on the sensed atmospheric temperature to account for moisture influences and also uses a heater to provide condensation protection. In at least some embodiments, this solution requires additional temperature corrections to compensate for the influence of moisture and depends on precise temperature measurements, which helps to make handling the sensor and evaluating the sensor data complex. In addition, the system does not offer complete protection against condensation, as there is no active evaporation of condensate, which could limit reliability in moist environments. Additionally, the solution comprises individual sensors and components that must be manually assembled, complicating the manufacturing process and potentially limiting production reproducibility, which may also lead to lower production numbers.

DE 10 2015 207 879 A1 describes a sensor device which, by means of a heated sealing element, helps enable improved, precise measurements of fluid media while minimizing condensation and icing. The sensor device may have several disadvantages due to its design. The increased energy consumption due to the need to heat the sealing element may be problematic in energy-critical applications. In addition, the integration of heating elements increases system complexity. Without a cover element, the membrane is exposed to environmental influences such as dust and moisture outside the cavity, which may impair its functionality and longevity. In addition, there is a risk to the membrane in the event of pressure surges if it divides the space and is impermeable to the medium, which could lead to damage or rupture of the membrane. These factors may affect the reliability and operational efficiency of the sensor device.

From DE 10 2018 119 212 A1 a sensor device with two pellistor elements is known. The sensor device comprises a common heating element and two separate temperature sensor elements arranged on the two pellistor elements and connected to an electronic arrangement. A disadvantage of this sensor device could be that gas mixtures cannot be measured in a differentiated manner. For this purpose, it is proposed to provide a catalyst element which is to be part of the pellistor elements. The sensor device with the two pellistor elements requires a total of four membranes and a heating element and a temperature measuring element for each membrane, helping to make its construction relatively complex and expensive. In addition, this increases the geometric dimensions of the sensor device. The multiple membranes present may not behave linearly in the event of drifts, contamination, condensate or other phenomena. The sensor device may be susceptible to external influences such as flow, pressure, drift, contamination of the sensor surfaces and other disturbing factors. Verification and calibration of such multi-component systems are complex.

WO 2022/106239 A1 discloses a sensor for detecting at least one property of a fluid medium in a measuring space. A sensor element of the sensor has a heatable membrane and a measuring bridge. Based on the electrical resistance of the measuring bridge, the sensor may measure the pressure of the fluid medium. Pressure measurement may be made possible by a micromechanical membrane dividing the gas medium into two parts and the pressure difference mechanically deforming the membrane. In the event of high pressure differences, the sensor may be destroyed. Pressure changes in the fluid medium influence the measurement result.

At least some embodiments disclosed herein at least partially overcome one or more disadvantages of the prior art. In at least some embodiments disclosed herein, a sensor element and a sensor as well as a method are to be found with which the thermal conductivity of a fluid medium, for example, a gas mixture, is able to be accurately measured. In at least some embodiments disclosed herein, a device in the form of a sensor element and a sensor is described, as well as a method for accurately measuring the thermal conductivity of a fluid medium, for example, a gas mixture, especially in the case of high moisture content or condensate. In at least some embodiments disclosed herein, the hydrogen concentration, the hydrocarbon gas concentration or the helium concentration in a gas mixture is accurately measurable despite the possibility of containing gaseous water. In at least some embodiments disclosed herein, the structure of the sensor element and the sensor is simple and cost-effective to manufacture. In at least some embodiments disclosed herein, a sensor element has low susceptibility to failure and high chemical resistance. In at least some embodiments disclosed herein, a sensor element has a structure that is relatively maintenance-free or low-maintenance.

The subject matter claimed herein relates to the above-described sensors, associated structures and methods, and the like.

In at least some embodiments, a sensor element is configured for measuring thermal properties of a fluid medium, for example, for measuring the thermal conductivity and/or for determining the hydrogen gas content, the hydrocarbon gas content or the helium content of the fluid medium, the sensor element comprising a membrane, a cover and a frame. In at least some embodiments, the frame surrounds the membrane on at least two sides. In at least some embodiments, the membrane is separated from the frame in regions by recesses. In at least some embodiments, the membrane is cantilevered and is connected to the frame via strips. In at least some embodiments, the frame supports the membrane via the strips, the sensor element further comprising at least one electrical resistance element in the form of at least one conductor path on the frame and an electrical heating element on the membrane in the form of a conductor path on the membrane. In at least some embodiments, the electrical heating element is electrically contacted via connecting conductor paths. In at least some embodiments, the connecting conductor paths run from the frame via at least one of the strips to the membrane. In at least some embodiments, the frame has an electrical heating element in the form of an external conductor path on the frame. In at least some embodiments, the electrical heating element of the frame surrounds the at least one electrical resistance element on the frame on the outside on at least two sides or on at least three sides.

In at least some embodiments, the sensor element is configured to detect the proportion of a hydrocarbon gas, an alkane gas or a He proportion or an H₂ proportion in a measurement gas, designed to detect an H₂ proportion in a measurement gas. The measurement gas is a gas mixture.

In at least some embodiments, the recesses are arranged or formed in the membrane, arranged or formed at the transition of the membrane to the frame.

In at least some embodiments, the connecting conductor paths are conductor paths that electrically contact the electrical heating element. In at least some embodiments, the connecting conductor paths are realized by metallization.

In at least some embodiments, the fluid medium is a gas or a gas mixture.

In at least some embodiments, the sensor element is a silicon-based micromechanical sensor.

In at least some embodiments, the sensor element is constructed as a MEMS.

In at least some embodiments, the membrane and the electrical heating element on the membrane do not have any catalytic or electrochemical coatings. In at least some embodiments, this helps improve the stability and longevity (durability) of the sensor element and helps prevent distortion of the measurement result due to reactions on the catalytic and/or electrochemical layer, which may be caused, for example, by contamination effects of the catalytic or electrochemical coatings. Furthermore, in at least some embodiments, a measurement is able to be carried out without additional fluid components, for example, oxygen.

Furthermore, in at least some embodiments, the two sides of the membrane are connected to each other via the recesses in a fluid-permeable or gas-permeable manner. In at least some embodiments, this helps prevent a pressure difference from building up between the upper side and lower side of the membrane, which would place mechanical stress on the membrane and the strips. In at least some embodiments, this helps enable the membrane and the strips to be constructed in a delicate manner, which helps to enable higher membrane temperatures and larger temperature differences between the membrane and the frame, which in turn helps improve the sensitivity of the sensor element. In at least some embodiments, a measurement is able to be carried out at high pressures without the membrane being destroyed or damaged by the pressure difference between the upper side and lower side.

In at least some embodiments, the at least one electrical resistance element on the frame and the electrical heating element on the membrane do not form a common conductor path. In at least some embodiments, the at least one electrical resistance element on the frame and the electrical heating element on the membrane are not directly electrically connected to one another. In at least some embodiments, the at least one electrical resistance element on the frame and the electrical heating element on the membrane form separate conductor paths which are supplied with an electrical voltage separately from one another.

The thermal properties of the fluid medium or gas are in at least some embodiments the thermal conductivity of the fluid medium or gas.

In at least some embodiments, the hydrogen gas content and the helium content of a fluid medium or gas mixture is calculable from the thermal conductivity of the fluid medium or the gas mixture. In at least some embodiments, the determination of the hydrogen content, the hydrocarbon gas content and the helium content provides a method for determining a thermal property of the fluid medium or gas, for example, using the thermal properties of a fluid medium or gas and a correlation to its composition.

At least some embodiments relate to a sensor element for measuring thermal properties of a fluid medium, comprising a membrane and a frame which surrounds the membrane on at least two sides. In at least some embodiments, the membrane is partially separated from the frame by recesses, the membrane is cantilevered and is carried by the frame via strips, the sensor element further comprising at least one electrical resistance element on the frame and an electrical heating element on the membrane. In at least some embodiments, the electrical heating element is electrically contacted via connecting conductor paths which run from the frame via at least one of the strips to the membrane.

In at least some embodiments, the membrane is heatable with the electrical heating element and the temperature of the membrane is able to be set via the electrical resistance of the electrical heating element. In at least some embodiments, the sensor element has an adjustable heating device for heating the electrical heating element and a membrane temperature setting device for setting the temperature of the membrane. In at least some embodiments, the membrane temperature setting device is configured to determine the electrical resistance of the electrical heating element.

In at least some embodiments, the membrane is heated with the same electrical heating element that is used to set the temperature of the membrane. In at least some embodiments, this arrangement helps reduce a time lag between setting and heating. In at least some embodiments, a compact design is provided that helps operate of the temperature of the membrane with less power.

In at least some embodiments, the recesses reduce the thermal stresses and heat flow from the membrane to the frame as a solid body. In at least some embodiments, this helps enable the temperature of the membrane to be set higher, and at the same time the measurement sensitivity and accuracy is able to be increased. In at least some embodiments, heat dissipating flow from the membrane is shifted to the heat dissipating flow through the fluid medium.

In at least some embodiments, a certain portion of a current is used to jointly heat the frame and the electrical heating element, and at least the electrical heating element on the membrane is additionally heated independently with a separate current.

Furthermore, in at least some embodiments, the temperature of the frame is able to be measured with the at least one electrical resistance element. In at least some embodiments, the sensor element has a frame temperature measuring device for measuring the temperature of the frame. In at least some embodiments, the frame temperature measuring device is configured to determine the electrical resistance of the at least one electrical resistance element.

In at least some embodiments, the measured temperature of the frame is used for thermostatic control of the frame, in which a deviation between the target and actual value is measured and brought to the target temperature by increasing or helping to reduce the heating output.

Furthermore, in at least some embodiments, the membrane and the frame are able to be heated independently of each other.

In at least some embodiments, this helps enable the temperature of the frame as a heat sink and the temperature of the membrane as a heat source to be set independently of each other, allowing the sensor element to be operated in a desired temperature range. In at least some embodiments, this is based on the thermal conductivity λ(T) being a temperature-dependent component with different gradients for different gases. If the influence of temperature is not only compensated but actively controlled, firstly a reproducible measurement behavior is obtained and secondly the difference in thermal conductivity between different gases in the fluid medium is able to be varied by flexible choice of the operating temperature, thereby increasing or decreasing the sensitivity between the gases to be detected. In at least some embodiments, the sensor element is able to be operated in this way in a temperature range around 200° C., which helps enable the measurement of the thermal conductivity (heat conductivity) of a gas in the background gas with a wide range of ratios of the components of the background gas, such as air, and/or nitrogen and/or oxygen and/or water vapor mixture, and thus helps reduce errors in the measurement results due to changes in the concentrations of individual components in the background gas. In at least some embodiments, the heated frame helps to prevent condensation on the sensor, for example, when the frame is heated to an excess temperature above ambient temperature. In at least some embodiments, the frame has a temperature, during operation of the sensor element, that is higher than the dew point temperature of the existing or possible condensate.

In at least some embodiments, the strips and the membrane consist of or include the same material and are designed as a single piece. In at least some embodiments, the membrane and the strips consist of or include silicon nitride or silicon oxide.

In at least some embodiments, the strips are made of silicon nitride or silicon oxide, and the membrane and the strips are coated with metal for a conductor path and/or coated with a protective layer of silicon oxide, silicon nitride or aluminum oxide or similar ceramics. In at least some embodiments, the connecting conductor paths run over the strips and the conductor paths of the electrical heating element on the membrane.

In at least some embodiments, the strips and the membrane are made of the same material and are designed as a single piece, which helps simplify the structure of the sensor element. In at least some embodiments, the recesses and thus the shape of the strips are created by etching.

In at least some embodiments, the frame consists of silicon or of a ceramic, for example, of silicon or of an aluminum oxide ceramic.

In at least some embodiments, the sensor element is built on a silicon wafer, on a ceramic block or on an aluminum oxide block. In at least some embodiments, the frame is made of silicon.

In at least some embodiments, the at least one electrical resistance element is realized by at least one metallic conductor path on the frame. In at least some embodiments, the at least one metallic conductor path consists of a metal or a metal alloy with a temperature coefficient of electrical resistance of at least 3 10⁻³ l/K or of nickel. In at least some embodiments, the electrical heating element is realized by a metallic conductor path on the membrane. In at least some embodiments, the metallic conductor path consists of a metal or a metal alloy with a temperature coefficient of at least 3 10⁻³ l/K or of nickel.

In at least some embodiments, such conductor paths help to enable determination of the electrical resistance or the power consumed. In at least some embodiments, for conductor paths made of these materials, for example nickel, the temperature dependence of the resistance is high, which helps enable accurate measurement of the temperature using the resistance.

In at least some embodiments, the conductor paths of the electrical heating element and of the at least one electrical resistance element are produced using a thin-film technology on the membrane or on the frame.

In at least some embodiments, the membrane is not arranged in an edge region of the sensor element and is surrounded on all sides by the frame, and the membrane is arranged in a central region of the sensor element.

In at least some embodiments, this helps protect the membrane on the outside of the frame from mechanical damage.

In at least some embodiments, the sensor element has a cover. In at least some embodiments, the cover delimits a gas volume around the membrane that is permeable to the environment and connected to the gas volume.

In at least some embodiments, the cover helps to ensure that the membrane remains unaffected by the disruptive influences of a gas or fluid flow. In at least some embodiments, the cover forms a closed, small volume which, in combination with the heating structure on the frame of the sensor, creates a vapor pressure in the volume and helps prevent the penetration of condensate to the measuring membrane and helps prevent damage to the membrane. In at least some embodiments, the frame structure runs transversely in the gas channel, which in at least some embodiments leads to a temperature hotspot on the way to the measuring membrane, since in this region the cover does not rest directly on the heating structure and emits little heat.

In at least some embodiments, the cover defines the internal volume and the distances above the membrane to the heat sink, which helps increase the reproducibility and response time of the thermal conductivity measurement.

In at least some embodiments, the cover helps to define a fluid channel or gas channel accessible from one side, which has an opening. In at least some embodiments, the fluid medium or the gas reaches the membrane through the fluid channel or gas channel, for example, by diffusion to the membrane. In at least some embodiments, the opening of the fluid channel or the gas channel is covered by a filter, for example, by a sintered metal filter. In at least some embodiments, the fluid channel or gas channel is open or accessible on a side of the sensor element which is arranged opposite contact fields for electrically contacting the sensor element.

In at least some embodiments, the cover consists of or includes silicon or a ceramic, for example, of an aluminum oxide ceramic. In at least some embodiments, the cover is made of silicon.

In at least some embodiments, the cover helps enable a reproducible measurement of the thermal conductivity of the fluid medium with the sensor element. In at least some embodiments, the cover also helps reduce the power consumption of the sensor. In at least some embodiments, the surfaces of the sensor element are protected from dust and condensate. In at least some embodiments, the sensor element with the cover is relatively immune or less susceptible to contamination phenomena.

In at least some embodiments, the cover is flush with the frame so that together they form a flush surface. In at least some embodiments, this design helps the gas channel to be flush when installing the sensor or sensor element in a housing, thus helping to reduce the dead volume to nearly zero. In at least some embodiments, a flat filter is installed or attached flush to the flush surface. In at least some embodiments, the dead volume is the space between the sensor or sensor element and the surrounding components, such as a sintered metal filter. In at least some embodiments, the dead volume is limited to the internal volume between the frame and the support surface. In at least some embodiments, the flush connection helps enable the use of a filter or a sintered metal filter for protection against flow, explosion and dust without any gaps.

In at least some embodiments, the sensor element has two electrical resistance elements in the form of two conductor paths as the at least one electrical resistance element on the frame. In at least some embodiments, the two electrical resistance elements surround the membrane on two opposite sides, so that the membrane is arranged between the two electrical resistance elements.

In at least some embodiments, the two electrical resistance elements are arranged symmetrically to the membrane. In at least some embodiments, this design helps allow for representative temperature determination at two locations on the sensor. In at least some embodiments, there are different temperature sources on the sensor and a temperature gradient exists across the sensor element. In at least some embodiments, there is a symmetrical arrangement of the electrical resistance elements, and there are two identical, representative temperature measuring points.

In at least some embodiments, the two electrical resistance elements are used for different applications. In at least some embodiments, while one uses the information to provide the required electrical power for temperature setting, the other's information is used in an analog circuit to compensate for the temperature influence on the accuracy of the measurement. In at least some embodiments, the combination helps enable a stable signal.

In at least some embodiments, the sensor element has a cantilevered wire as a reference resistor. In at least some embodiments, the cantilevered wire is connected to the frame at two ends and suspended from the frame and is spatially separated from the frame in some regions by recesses. In at least some embodiments, the cantilevered wire is surrounded by the cover, which is firmly connected to the frame. In at least some embodiments, the cover is spatially spaced from the cantilevered wire at least in some regions. In at least some embodiments, the cover is constructed at least in some regions as a gas-permeable filter.

In at least some embodiments, the cantilevered wire is used as a reference. In at least some embodiments, this helps enable influences such as pressure changes and/or temperature changes and/or flow changes to be compensated. In at least some embodiments, the cantilevered wire is arranged in a fluid volume separate from the fluid volume of the membrane. If the cantilevered wire has its own gas channel or fluid volume, heat is less able to be transferred from the membrane as a heat source to the cantilevered wire as a reference by convection of the gas or fluid medium.

In at least some embodiments, a sensor and a sensor element with a cantilevered wire enable, among other things, the compensation of pressure influences on the measuring signal. The sensor according to at least one embodiment of the disclosure is usable for pressures from 300 mbar up to 1000 bar and theoretically even beyond. The pressure dependence of the thermal conductivity measurement is due to the mean free path of the gas molecules and the geometry of the sensor. For measuring the thermal conductivity or the content of the desired molecules in the fluid medium, the cantilevered wire with a relatively small surface area is able to be used for a reference measurement. A comparison of the reference measurement with the cantilevered wire and a measurement with a measuring membrane that has a relatively large membrane area shows different results in heat transfer. These differences are mainly due to the different geometric designs of the two sensor elements, although the underlying measurement method remains the same. Such measurements are relevant and well suited to compensate for influences caused by changing pressure and temperature of the fluid medium. In at least some embodiments, the temperature is kept constant during the measurement so that the reference through the exposed wire is able to be used to compensate for pressure changes.

In at least some embodiments, the cover surrounding the cantilevered wire separates the cantilevered wire from a fluid volume of the sensor element surrounding the membrane.

In at least some embodiments, the membrane is surrounded by the cover on at least two sides to the frame and above and below the membrane. In at least some embodiments, the cover is constructed at least in regions as a gas-permeable filter and/or at least in regions made of silicon.

In at least some embodiments, this helps to help to provide that the membrane remains unaffected by the disruptive influences of a gas or fluid flow. In at least some embodiments, the geometry of the cover defines a fixed distance between the heat source on the membrane and the heat sink formed by the cover and the frame. In at least some embodiments, this helps enable reproducibility of the measurement result.

In at least some embodiments, the frame has an electrical heating element in the form of an outer conductor path on the frame. In at least some embodiments, the electrical heating element of the frame frames the at least one electrical resistance element on the frame to the outside on at least two sides or on at least three sides. In at least some embodiments, the outer conductor path on the frame is wider than the conductor paths of the at least one electrical resistance element on the frame and of the electrical heating element on the membrane.

In at least some embodiments, the frame is heated or temperature-controlled independently and using the electrical heating element on the frame, while the at least one electrical resistance element on the frame is used to determine the temperature of the frame. In at least some embodiments, this helps enable the temperature of the membrane to be set by a heating output in the electrical heating element on the membrane and the temperature of the frame to be set by a heating output in the electrical heating element on the frame.

In at least some embodiments, an exposed part of the outer conductor path of the electrical heating element of the frame runs in a fluid channel which leads to a fluid volume surrounding the membrane. In at least some embodiments, the exposed part of the outer conductor path runs transversely to the fluid channel and/or the fluid channel is formed in the cover which encloses the membrane. In at least some embodiments, the cover covers the other regions of the outer conductor path.

In at least some embodiments, this helps to reduce or eliminate flow influences during measurement. In addition, the fluid medium reaching the membrane is able to be preheated.

In at least some embodiments, the at least one electrical resistance element on the frame and the electrical heating element on the membrane, and if present also the electrical heating element of the frame, are electrically contacted via contact fields on the frame. In at least some embodiments, the contact fields are formed by widened regions of the conductor paths and/or the contact fields are all arranged on one side of the frame.

In at least some embodiments, this helps enable the sensor element to be electrically contacted and installed. At the same time, a compact design is possible, which results in a shorter reaction time of the sensor element.

In at least some embodiments, the membrane has an area of at most 20 mm², has an area of at most 1 mm², has an area of at most 0.5 mm², very has an area of at most 0.1 mm².

In at least some embodiments, this helps enable a compact design that achieves a shorter response time of the sensor element. In addition, the electrical connection power of the sensor element is able to be kept low.

In at least some embodiments, the frame with the membrane in the plane of the membrane has an outer diameter of at most 10 mm, an outer diameter of at most 3 mm, an outer diameter of at most 2 mm.

In at least some embodiments, this helps enable a compact design that achieves a shorter response time of the sensor element. In at least some embodiments, the electrical connection power of the sensor element is able to be kept low.

In at least some embodiments, cover is provided with heating.

In at least some embodiments, the cover is heated with the electrical heating element on the frame.

In at least some embodiments, the cover defines at least one gas inlet opening of the sensor element. In at least some embodiments, the conductor path of the electrical heating element on the frame is exposed in the at least one gas inlet opening.

In at least some embodiments, one or more of the sensor features help to prevent the formation of condensate in the measuring volume contained by the cover and thus helps to reduce errors in the measurement result and/or damage to the sensor element. In at least some embodiments, the cover functions as a heater. In at least some embodiments, the structure on the frame of the sensor functions as a heater.

In at least some embodiments, a sensor is useable for determining thermal properties of a fluid medium, for example, for determining the thermal conductivity and/or the hydrogen gas content, the hydrocarbon gas content or the helium content of a fluid medium, the sensor comprising a sensor element according to at least one embodiment of the disclosure or previously described. In at least some embodiments, the membrane is able to be heated with the electrical heating element on the membrane independently of the temperature of the frame or the membrane with the electrical heating element on the membrane and the frame independently of each other. In at least some embodiments, the temperature of the membrane with the electrical heating element on the membrane and the temperature of the frame is able to be measured with the at least one electrical resistance element. In at least some embodiments, the temperature of the membrane with the electrical heating element on the membrane and the temperature of the frame is able to be regulated based on the measurement of the temperatures. In at least some embodiments, the heating powers required for this are determined with the sensor in order to draw conclusions about the thermal properties of the fluid medium, in order to determine the thermal conductivity and/or the hydrogen gas content, the hydrocarbon gas content or the helium content of the fluid medium.

In at least some embodiments, the sensor is used directly for measurement and incorporates the features of the sensor element.

In at least some embodiments, the sensor has a Wheatstone bridge circuit which is connected to the electrical heating element on the membrane in such a way that the heating power of the membrane is able to be regulated via the Wheatstone bridge circuit.

In at least some embodiments, this helps enable the measurement to be carried out with high reaction speed and stably.

In at least some embodiments, the Wheatstone bridge circuit is a self-regulating Wheatstone bridge circuit.

In at least some embodiments, the membrane is able to be heated to temperatures of at least 200° C. during operation of the sensor and/or the frame is able to be heated to at least 130° C. during operation of the sensor. In at least some embodiments, the membrane is able to be heated to temperatures of at least 220° C. during operation of the sensor and/or the frame is able to be heated to at least 150° C. during operation of the sensor. In at least some embodiments, the frame is able to be heated to at least 180° C. during operation of the sensor.

In at least some embodiments, this helps to help to provide that the measurement is carried out in a temperature range that lies in the quasi-binary range and thus the hydrogen gas content, the hydrocarbon gas content or the helium content is able to be determined independently of the water vapor or water gas content in the fluid medium or in the gas mixture. In at least some embodiments, this helps to make the determination of the hydrogen gas content in a gas such as air, oxygen or nitrogen more accurate, especially when the water vapor content in the gas is different.

Using the same method as for water vapor in oxygen or air, but at different temperatures, the intersection points of the thermal conductivities can also be determined for other gas pairs, such as carbon dioxide and argon or nitrogen and ammonia.

In at least some embodiments, a method is provided for determining at least one thermal property of a fluid medium, for example, the thermal conductivity and/or the hydrogen content, the hydrocarbon gas content or the helium content of the fluid medium, the method comprising the following steps:
A) heating a membrane with an electrical heating element in the form of a conductor path arranged on the membrane. In at least some embodiments, the membrane is connected to a frame via strips and is held by the frame and wherein the membrane is surrounded by a defined volume of the fluid medium within a cover;
B) transferring heat from the membrane and the electrical heating element to the fluid medium surrounding the membrane;
C) transferring heat from the fluid medium to the frame. In at least some embodiments, the frame has a lower temperature than the membrane;
D) determining the temperature of the membrane and the frame;
E) controlling or regulating the temperature of the membrane by regulating the electrical power for heating the membrane;
F) determining the thermal property of the fluid medium, for example, the thermal conductivity and/or the hydrogen content, the hydrocarbon gas content or the helium content of the fluid medium, by the electrical power required to regulate the temperature of the membrane and by the known temperature difference between the membrane and the frame; and
G) controlling or regulating the temperature of the frame.
   Step G) provides:
G) controlling or regulating the temperature of the frame. In at least some embodiments, in step G) the temperature of the frame is controlled or regulated by regulating the electrical power for heating the frame.

In at least some embodiments, this helps keep the frame at a defined temperature, helping to make the measurement more reproducible and stable.

In at least some embodiments, the temperature of the membrane is kept above 200° C. and the temperature of the frame is kept below 200° C.

In at least some embodiments, this helps to help to provide that the measurement is carried out in a temperature range that lies in the quasi-binary range and thus the measuring gas content, for example, the hydrogen gas content or the hydrocarbon gas content or the helium content is able to be determined independently of the water vapor or water gas content in the fluid medium or in the gases such as air, oxygen or nitrogen. In at least some embodiments, this helps to make the determination of the measuring gas content, for example, hydrogen gas content in a gas at different water gas contents in the gas such as air, oxygen or nitrogen more accurate.

In at least some embodiments, the membrane and the frame are part of a sensor. In at least some embodiments, the sensor has a constant and known geometry and a known heating behavior and wherein the sensor is calibrated before the measurement in order to calculate the thermal property of the fluid medium, for example, the thermal conductivity and/or the hydrogen content, the hydrocarbon gas content or the helium content of the fluid medium, from the known geometry of the sensor and from the known or measured temperature difference between the membrane and the frame and the electrical power required to heat the membrane.

In at least some embodiments, this helps enable the thermal properties of the fluid medium to be determined more reproducibly and precisely.

In at least some embodiments, the sensor is calibrated at least once during the sensor manufacturing process.

In at least some embodiments, the geometry of the sensor is determined by its dimensions and shape.

In at least some embodiments, the method is carried out with a sensor element according to at least one embodiment of the disclosure or previously described, or with a sensor according to at least one embodiment of the disclosure or previously described.

In at least some embodiments, the method benefits from the properties of the sensor element or the sensor.

In at least some embodiments, the temperature of the membrane is controlled or regulated at least 5° C. and at most 200° C. above the temperature of the frame, the temperature of the membrane is controlled or regulated at least 10° C. and at most 150° C. above the temperature of the frame, the temperature of the membrane is controlled or regulated at least 20° C. and at most 100° C. above the temperature of the frame.

In at least some embodiments, the temperature of the membrane is kept at least 201° C. and at most 350° C., at least 210° C. and at most 300° C., at least 220° C. and at most 260° C.

In at least some embodiments, the temperature of the frame is kept at least at 135° C. and at most 199° C., at least 150° C. and at most 195° C., at least 180° C. and at most 190° C.

In at least some embodiments, the temperature of the membrane is controlled or regulated in such a way or the temperature of the membrane and the temperature of the frame are controlled or regulated in such a way that the average value of the temperature of the frame and the temperature of the membrane is at least 180° C. and at most 220° C., at least 190° C. and at most 210° C., at least 195° C. and at most 205° C.

In at least some embodiments, one or more of these measures help to provide that the measurement is carried out in a temperature range that lies in the quasi-binary range and thus the measuring gas content, for example, the hydrogen gas content or the hydrocarbon gas content or the helium content, is able to be determined independently of the water vapor or water gas content in the fluid medium or in the gas or gases such as air, oxygen or nitrogen (as the fluid medium). In at least some embodiments, this helps to make the determination of the hydrogen gas content, the hydrocarbon gas content or the helium content in a gas more accurate when there are different water gas contents in the gases air, oxygen or nitrogen.

In at least some embodiments, a temperature of a separate volume in the region of the frame is measured with a cantilevered wire. In at least some embodiments, the measured temperature of the cantilevered wire is used for correction when determining the thermal property of the fluid medium, for example, the thermal conductivity and/or the hydrogen content, the hydrocarbon gas content or the helium content of the fluid medium, in step F).

In at least some embodiments, the heating power Q and the measurement are determined by the defined geometry and the thermal conductivity (as a function of temperature) and thus calculated as in the following Formula 2:

$$Q = λ ( T ) ( A ( T q - T s ) ) / d \qquad ( 2 )$$

wherein, in Formula 2,
λ(T) is the function of thermal conductivity as a function of temperature T;
A is the area of the heat source (defined freely suspended area that is electrically heated by the heating element),
d a defined distance between the heat source and the heat sink (the walls of the measuring chamber formed by the frame). In at least some embodiments, the walls are heated to a high temperature;
Tq is the temperature of the heat source (the membrane); and
Ts is the temperature of the heat sink (the frame).

In at least some embodiments, the area of the cantilevered wire or the cantilevered resistance wire is smaller than the area of the cantilevered membrane. In at least some embodiments, this results in two thermal conductivity measurements with different areas.

For the same gas, the two measuring elements have different interactions with the pressure. Through a correlation measurement, the pressure influences are able to be compensated digitally (using a mathematical model) or in an analog manner (using a differential circuit).

In at least some embodiments, this helps enable the cantilevered wire, which is a cantilevered resistance wire, to be used as a reference. In at least some embodiments, this helps enable influences such as pressure changes or flow changes of the fluid medium to be measured and compensated for. In at least some embodiments, the cantilevered wire is arranged in a fluid volume separate from the fluid volume of the membrane. In at least some embodiments, the cantilevered wire has its own gas channel or fluid volume, which helps to reduce or prevent heat transfer from the membrane (heat source) to the cantilevered wire (reference) by convection of the gas or fluid medium.

In at least some embodiments, the same electrical heating element is used on the membrane to determine the temperature of the membrane in step D) as is used to heat the membrane in step A).

In at least some embodiments, this helps enable a compact membrane design to be achieved, which helps improve the reproducibility and stability of the measurement and helps increase the operating temperature of the membrane.

In at least some embodiments, the temperature of the membrane is kept constant by a first self-regulating Wheatstone bridge circuit. In at least some embodiments, the electrical heating element on the membrane is part of the first self-regulating Wheatstone bridge circuit, and/or the temperature of the frame is kept constant by a second self-regulating Wheatstone bridge circuit or by a digital or computer-controlled controller. In at least some embodiments, at least one electrical resistance element on the frame and/or an electrical heating element on the frame is part of the second self-regulating Wheatstone bridge circuit.

In at least some embodiments, this helps enable the temperature of the membrane to be controlled in a simple manner and at the same time helps to make the electrical power required therefor easier to measure, which helps for determining the thermal properties of the fluid medium. In at least some embodiments, the method further comprises the following step H): H) increasing or controlling or regulating the temperature of the fluid medium in the region of a gas inlet to the membrane in the cover and/or increasing or controlling or regulating the temperature of the cover. In at least some embodiments, the cover delimits a gas volume around the membrane that is connected to the environment in a gas-permeable manner, by regulating the electrical power for heating the frame or a separate electrical resistance heating device.

In at least some embodiments, this helps prevent the formation of condensate in the measuring volume contained by the cover and thus an error in the measurement result or damage to the measuring structure.

In at least some embodiments, the frame is controlled or set to a temperature higher than the dew point temperature of the existing or possible condensation in the gas under investigation.

In at least some embodiments, this helps prevent condensate from forming on the membrane and destroying it.

In at least some embodiments, the electrical heating element on the membrane carried by the frame in combination with the at least one resistance element on the frame of the sensor element helps to provide a measuring arrangement with which heat transfer from the membrane to the frame through the fluid medium surrounding the sensor element is able to be determined precisely, helping to provide an accurate determination of the composition, for example, the hydrogen content, the hydrocarbon gas content or the helium content. In at least some embodiments, this arrangement helps enable the temperature at which the sensor element operates to be kept in the range of 200° C., which helps to determine the hydrogen content, the hydrocarbon gas content or the helium content of a gas independently of the water vapor content. In at least some embodiments, the electrical heating element is not only used to heat the membrane, but also to determine the temperature of the electrical heating element on the membrane.

If a measured component consists of several gases that behave like a single gas with regard to their thermal conductivity, a binary measurement of the gas mixtures is referred to as of a quasi-binary gas mixture. An example is the case when these components remain permanently in unchanged proportions, such as in dry atmospheric air, or when the components of this mixture have the same thermal conductivity.

Of all gases, hydrogen has by far the highest thermal conductivity. The gas with the next highest thermal conductivity is helium. This, together with the high stability and ease of use, helps to make hydrogen or helium measurement using the thermal conductivity method attractive.

In the sensor element according to at least one embodiment of the present disclosure, the membrane is suspended from thin strips. In at least some embodiments, this design helps decouple the membrane from the frame and helps prevent temperature deviations due to temperature leakage from the membrane through solid bodies. In at least some embodiments, this in turn helps provide a homogeneous distribution of heat on the membrane. In at least some embodiments, apart from the membrane, only a single conductor path is arranged on the membrane as an electrical heating element. In at least some embodiments, this involves using the electrical heating element as a temperature measuring element. In at least some embodiments, other parts are free of separate heating and the structure of the sensor element is simplified. At the same time, the strong thermal decoupling of the membrane from the frame helps to make it possible to use the electrical heating element as a temperature measuring element. The at least one electrical resistance element, on the other hand, is arranged on the frame so that it does not increase the mass of the membrane with the electrical heating element and simplifies the construction of the membrane with the electrical heating element. At the same time, this allows the frame to be brought to a temperature such that the measurement is able to be carried out at temperatures at which the moisture in a gas such as air or oxygen has no or at least a lesser negative influence on the determination of the hydrogen concentration or the helium concentration in the gas.

In at least some embodiments, the absence of a catalytic layer may lead to a loss of selectivity in the trace range, but at the same time results in a higher measurement accuracy of the thermal conductivity of the fluid medium and an elimination of a source of interference for the measurement of the thermal conductivity of the fluid medium, which is caused by chemical components of the fluid medium that chemically react with the catalytic layer. The lack of selectivity is advantageous in some measurement applications, since in this way the approach or approximation of a measurement of quasi-binary fluid mixtures or gas mixtures is able to be used or applied. In addition, the sensor element according to at least one embodiment of the disclosure is free of a catalytic layer and is not susceptible to contamination phenomena that are associated with a catalytic layer.

In at least some embodiments, the recesses help to provide that both sides of the membrane are within the same fluid medium. In at least some embodiments, this helps ensure that no pressure differences occur on the two sides of the membrane. In at least some embodiments, this helps prevent deformation of the membrane due to pressure differences. In at least some embodiments, this means that any distortion of the thermal conductivity measurement result due to deformation of the membrane and the electrical heating element arranged on the membrane is excluded or reduced. In this way, a higher measurement accuracy is able to be achieved with the sensor element. In addition, the sensor element according to at least one embodiment of the disclosure is operable under high pressures without being destroyed. In the sensor element according to at least one embodiment of the disclosure, the pressure changes only have an influence due to the change in the mean free path of the gas molecules. In at least some embodiments, this effect is small at normal and high pressures and is able to be neglected in most cases. The changes in pressure would destroy sensor elements and sensors without recesses and with a closed membrane at an earlier point in time than sensor elements and sensors according to at least one embodiment of the disclosure.

Due to the structure with the strips and the recesses, the heat dissipation from the membrane to the frame through the solid body is drastically reduced or almost non-existent. The structure of the sensor element according to at least one embodiment of the disclosure and of the sensor with strips according to at least one embodiment of the disclosure is therefore more robust, since thermal stresses are avoided within a surface of the membrane connected to the frame. The temperature difference between the heat source and heat sink can therefore be greater. In at least some embodiments, this results in better signal quality and signal amplitude. Furthermore, the membrane heating requires less power due to the better decoupling. In addition, the area of the chip (the frame) and the area of the membrane is able to be at least halved compared to conventional sensor elements without strips and recesses of the membrane to the frame. In the structure according to at least one embodiment of the disclosure, the chip or the frame has an area of, for example, 2 by 2 mm² and the membrane has an area of, for example, 0.3 by 0.3 mm² or less. A smaller membrane helps enable the measuring chamber to be made smaller. In at least some embodiments, this helps to make the gas exchange faster, especially if the gas in the measuring chamber is to be exchanged through diffusion processes. Alternatively or simultaneously, the depth or height of the measuring chamber is able to be selected to be larger, whereby the sensor element requires less power and the pressure dependence is reduced.

In general sensors and sensor elements with continuous membranes and without strips, the electrical heating elements on the membrane must be as far away as possible from the edge of the membrane in order to minimize heat dissipation. In contrast, in a sensor according to at least one embodiment of the disclosure, the entire membrane is able to be covered with the conductor paths of the electrical heating element. In at least some embodiments, this helps improve the homogeneity of the temperature distribution across the membrane.

In sensor elements and sensors according to at least one embodiment of the disclosure, the electrical contacts or contact fields and the opening of the measuring chamber are able to be spatially separated from one another. In at least some embodiments, this helps enable the contacts to be cast with durable material without closing the measuring chamber opening.

### BRIEF DESCRIPTION OF THE DRAWINGS

In the following, embodiments of the disclosure are explained with reference to ten schematically illustrated figures and a flow chart, without, however, limiting the disclosure. In particular:

FIG. 1 shows a schematic plan view of a first sensor element according to at least one embodiment of the disclosure;

FIG. 2 shows, at the top a schematic plan view of the sensor element according to FIG. 1 with a cover and at the bottom a schematic front view of the sensor element according to FIG. 1 with the cover;

FIG. 3 shows a schematic cross-sectional view of the section shown in FIG. 2 at the top along the dash-dot line A-A;

FIG. 4 shows a schematic plan view of a second sensor element according to at least one embodiment of the disclosure;

FIG. 5 shows, at the top a schematic plan view of the sensor element according to FIG. 4 with a cover and at the bottom a schematic front view of the sensor element according to FIG. 4 with the cover;

FIG. 6 shows a schematic cross-sectional view of the section shown in FIG. 5 at the top along the dash-dot line B-B;

FIG. 7 shows a perspective view of a sensor element according to at least one embodiment of the disclosure on a circuit board for electrical contacting;

FIG. 8 shows a schematic cross-sectional view of a head of a sensor according to at least one embodiment of the disclosure, in which the sensor element according to at least one embodiment of the disclosure is installed on the circuit board according to FIG. 7;

FIG. 9 shows a schematic cross-sectional view of a head of a further sensor according to at least one embodiment of the disclosure, in which the sensor element according to at least one embodiment of the disclosure is installed on the circuit board according to FIG. 7;

FIG. 10 shows the thermal conductivity in W/m K as a function of temperature in ° C. of gaseous hydrogen, water, air and oxygen; and

FIG. 11 shows the sequence of a method according to at least one embodiment of the disclosure for determining at least one thermal property of a fluid medium.

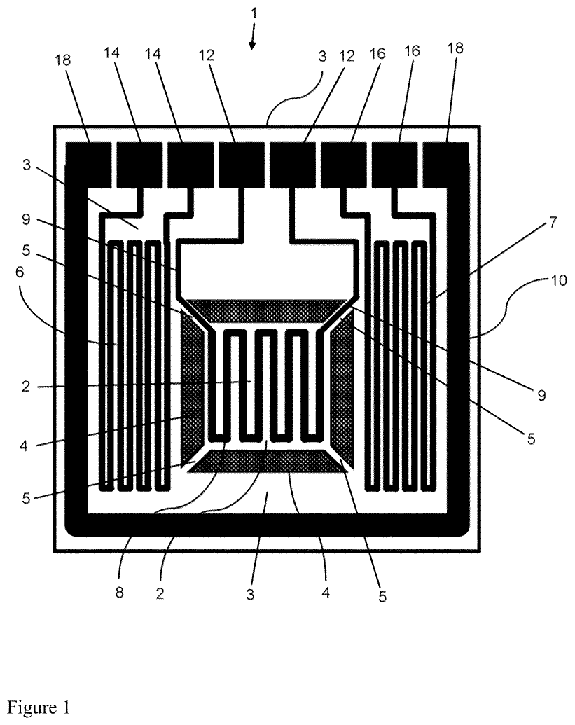

FIGS. 1 to 3 show various schematic views of a first sensor element 1 according to at least one embodiment of the disclosure.

### DETAILED DESCRIPTION

In at least some embodiments, a sensor element 1 has a membrane 2 and a frame 3. In at least some embodiments, the frame 3 is made of silicon or of a ceramic, for example, of an aluminum oxide ceramic. The membrane 2 is held by the frame 3 and is otherwise freely suspended. In at least some embodiments, the membrane 2 is made of silicon nitride. The membrane 2 is physically separated from the frame 3 in some regions by recesses 4. In at least some embodiments, to hold the membrane 2 on the frame 3, the membrane 2 is connected to the membrane 2 via strips 5. The recesses 4 are only bridged by the strips 5. The strips 5 are made of the same material as the membrane 2 itself.

Such a structure is able to be produced, for example, by first coating a silicon body with a silicon nitride layer. Subsequently, the recesses 4 are formed in the silicon nitride layer using known exposure and etching processes and the silicon body is removed below the part of the silicon nitride layer intended for the membrane 2. In at least some embodiments, this creates a freely floating membrane 2 which is connected to a frame 3 via strips 5 between the recesses 4.

The sensor element 1 according to FIG. 1 has a first electrical resistance element 6. In at least some embodiments, the first electrical resistance element 6 is arranged in the form of a conductor path on the frame 3 and is configured as a meandering conductor path. In at least some embodiments, the temperature of the frame 3 is measured with the first electrical resistance element 6. In at least some embodiments, the sensor element 1 also has a second electrical resistance element 7. In at least some embodiments, the second electrical resistance element 7 is arranged in the form of a conductor path on the side of the frame 3 opposite the membrane 2 and is designed as a meandering conductor path. In at least some embodiments, the temperature of the frame 3 is measured with the second electrical resistance element 7.

In at least some embodiments, the first electrical resistance element 6 and the second electrical resistance element 7 are made of nickel and are configured to heat the frame 3, at least in the region of the electrical resistance elements 6, 7, to a certain temperature, for example, to heat the silicon structure as frame 3 to a certain temperature. Alternatively or additionally, the electrical resistance elements 6, 7 are able to be used to measure the temperature of the frame 3. In at least some embodiments, the temperature of frame 3 is maintained to be lower than the temperature of membrane 2 during the measurement so that frame 3 acts as a heat sink.

An electrical heating element 8 is arranged on the membrane 2, which is able to be used both for heating and for measuring the temperature. The electrical heating element 8 is able to be designed as a meandering conductor path on the membrane 2. In at least some embodiments, the electrical heating element 8 is made of nickel. The electrical heating element 8 is electrically contacted via connecting conductor paths 9, so that the connecting conductor paths 9 electrically connect the electrical heating element 8 up to the frame 3. In at least some embodiments, the connecting conductor paths 9 is constructed from the same conductor paths as the electrical heating element 8. In at least some embodiments, the connecting conductor paths 9 are guided over two different strips 5, as shown in FIG. 1. In at least some embodiments, the connecting conductor paths 9 are routed via a single strip 5 (not shown).

In at least some embodiments, an outer electrical heating element 10 is arranged on the frame 3 for heating the frame 3. In at least some embodiments, the outer electrical heating element 10 frames the electrical resistance elements 6, 7 and the membrane 2 in the region of outer ends of the frame 3. In the embodiment described with respect to FIG. 1, a framing is seen on three sides of the sensor element 1. In at least some embodiments, the external electrical heating element 10 is configured as an external conductor path. In at least some embodiments, the outer electrical heating element 10 is made of nickel. In at least some embodiments, the outer electrical heating element 10 is realized by a conductor path that is wider than that of the electrical resistance elements 6, 7 and than that of the electrical heating element 8 on the membrane 2. In at least some embodiments, this helps enable a stronger current to be passed through the outer electrical heating element 10 at a lower voltage without fear of damage to the outer electrical heating element 10. In at least some embodiments, the frame 3 has a significantly higher heat capacity than the membrane 2, and a higher heating power is applied in the frame 3 than in the membrane 2. In at least some embodiments, the electrical resistance elements 6, 7 are also used to heat the frame 3 to a desired temperature.

In at least some embodiments, the electrical heating element 8 on the membrane 2 is electrically connected to contact fields 12 via the connecting conductor paths 9. In at least some embodiments, the first electrical resistance element 6 is electrically connected to contact fields 14. In at least some embodiments, the second electrical resistance element 7 is electrically connected to contact fields 16. In at least some embodiments, the outer electrical heating element 10 is electrically connected to contact fields 18. In at least some embodiments, the contact fields 12, 14, 16, 18 are made of the same material as the conductor paths that form the electrical resistance elements 6, 7, the electrical heating element 8, the connecting conductor paths 9 and the outer electrical heating element 10. In at least some embodiments, the contact fields 12, 14, 16, 18 are made of nickel. In at least some embodiments, the contact fields 12, 14, 16, 18 are wider or have a larger diameter than the conductor paths connected to them. In at least some embodiments, all contact fields 12, 14, 16, 18 are arranged on one side of the frame 3 on the frame 3.

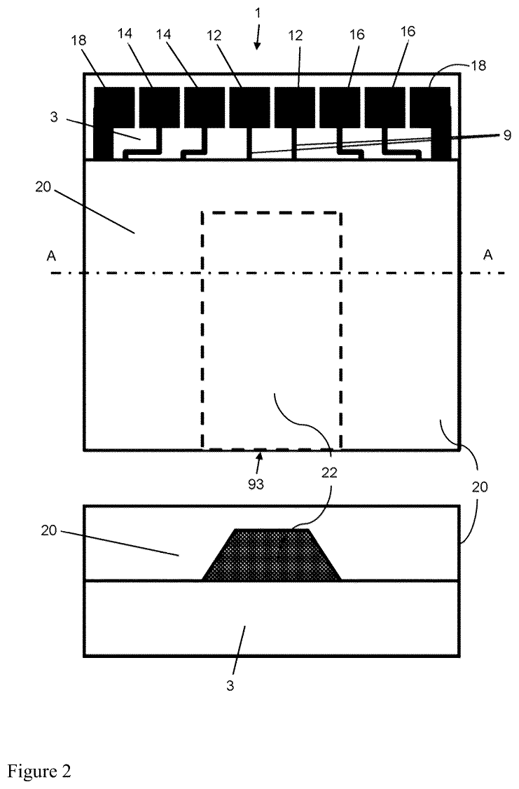

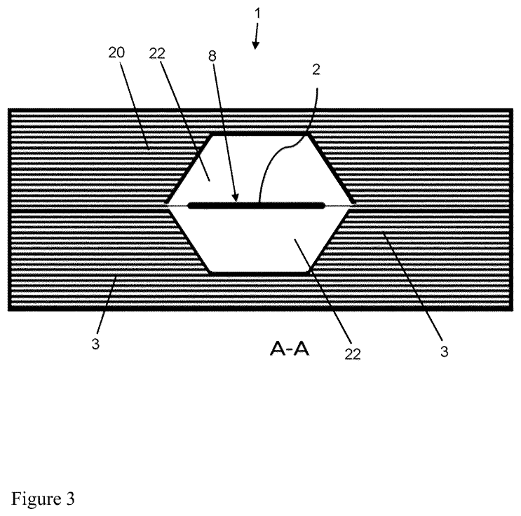

In at least some embodiments, the sensor element 1 shown in FIG. 1 includes one or more additional elements. In at least some embodiments, the sensor element 1 also has a cover 20 which encloses a fluid volume 22 or a gas volume around the membrane 2. In at least some embodiments, the fluid volume 22 or gas volume forms a measuring chamber for the fluid medium or gas to be measured. This is shown in FIGS. 2 and 3. The structure on the frame 3 and on the membrane 2 is identical to the sensor element 1 shown in FIG. 1. The cover 20 covers only the conductor paths, the membrane 2 and the upper side of the frame 3 shown in FIG. 1 except for the contact fields 12, 14, 16, 18 and adjacent regions, as shown in FIG. 2 above. The gas volume is accessible via a gas inlet opening 93. In at least some embodiments, the conductor paths on the frame 3 are exposed in the region of the gas inlet opening 93.

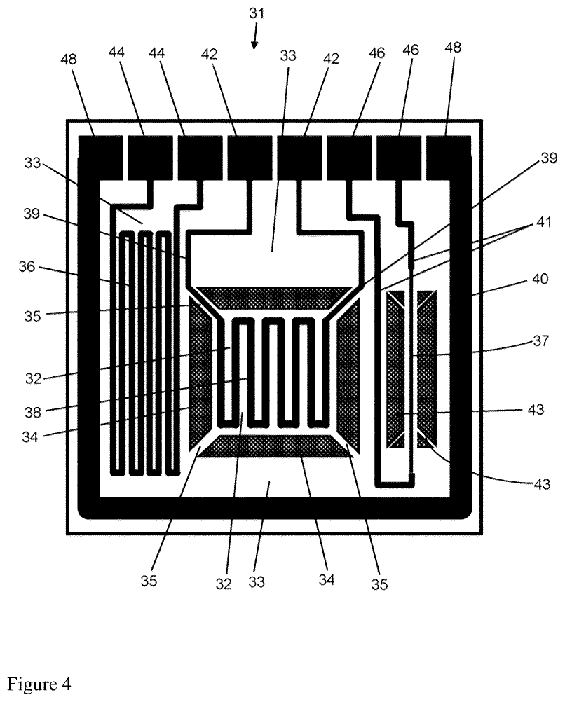

FIGS. 4 to 6 show various schematic views of a second sensor element 31 according to at least one embodiment of the disclosure. The sensor element 31 differs from the sensor element 1 according to FIGS. 1 to 3 primarily by the presence of a cantilevered wire 37, which is usable as a reference measuring point in order to improve the measurement result and to eliminate disturbing variables.

The sensor element 31 has a membrane 32 and a frame 33. In at least some embodiments, the frame 33 is made of silicon or of a ceramic, for example, of an aluminum oxide ceramic. In at least some embodiments, the membrane 32 is held by the frame 33 and is otherwise freely suspended. In at least some embodiments, the membrane 32 is made of silicon nitride. The membrane 32 is physically separated from the frame 33 in some regions by recesses 34. In at least some embodiments, to hold the membrane 32 on the frame 33, the membrane 32 is connected to the membrane 32 via strips 35. In at least some embodiments, the recesses 34 are only bridged by the strips 35. In at least some embodiments, the strips 35 are made of the same material as the membrane 32 itself.

In at least some embodiments, the such a structure is produced, for example, by first coating a silicon body with a silicon nitride layer. Subsequently, the recesses 34 are formed in the silicon nitride layer using exposure and etching processes and the silicon body is removed below the part of the silicon nitride layer intended for the membrane 32. In at least some embodiments, this creates a freely floating membrane 32 which is connected to a frame 33 via strips 35 between the recesses 34.

The sensor element 31 according to FIG. 4 has an electrical resistance element 36. In at least some embodiments, the electrical resistance element 36 is arranged in the form of a conductor path on the frame 33 and is configured as a meandering conductor path. In at least some embodiments, the temperature of the frame 33 is measured with the electrical resistance element 36. In at least some embodiments, the electrical resistance element 36 is used to heat the frame 33. In at least some embodiments, the sensor element 31 has a cantilevered wire 37 which is arranged cantilevered in a free space and which is spatially separated from the frame 33 via recesses 43. In at least some embodiments, the cantilevered wire 37 is connected to contact fields 46 on the frame 33 via connecting conductor paths 41 on the frame 33. In at least some embodiments, with the cantilevered wire 37, the temperature of a body with a small surface compared to the membrane 32 is measured as a reference value.

In at least some embodiments, the electrical resistance element 36 is made of nickel and is configured to heat the frame 33 to a certain temperature, for example, heat the silicon structure as frame 33 to a certain temperature.

In at least some embodiments, the cantilevered wire 37 is made of nickel. In at least some embodiments, this temperature of frame 33 is controlled to be lower than the temperature of membrane 32 during the measurement so that frame 33 acts as a heat sink.

An electrical heating element 38 is arranged on the membrane 32, and in at least some embodiments is used both for heating and for measuring the temperature. In at least some embodiments, the electrical heating element 38 is configured as a meandering conductor path on the membrane 32. In at least some embodiments, the electrical heating element 38 is made of nickel. The electrical heating element 38 is electrically contacted via connecting conductor paths 39, so that the connecting conductor paths 39 electrically connect the electrical heating element 38 up to the frame 33. In at least some embodiments, the connecting conductor paths 39 is constructed from the same conductor paths as the electrical heating element 38. In at least some embodiments, the connecting conductor paths 39 are guided over two different strips 35, as shown in FIG. 4. In at least some embodiments, the connecting conductor paths 39 are routed via a single strip 35 (not shown).

In at least some embodiments, an outer electrical heating element 40 is arranged on the frame 33 for heating the frame 33. In at least some embodiments, the outer electrical heating element 40 is configured to heat the electrical resistance elements 36, the cantilevered wire 37, the connecting conductor paths 41 and the membrane 32 in the region of outer ends of the frame 33. In the embodiment described with respect to FIG. 4, a framing is on three sides of the sensor element 31. In at least some embodiments, the outer electrical heating element 40 is configured as an external conductor path. In at least some embodiments, the outer electrical heating element 40 is made of nickel. In at least some embodiments, the outer electrical heating element 40 is realized by a conductor path that is wider than that of the electrical resistance element 36 and than the electrical heating element 38 on the membrane 32. In at least some embodiments, this helps enable a stronger current to be passed through the outer electrical heating element 40 at a lower voltage without destroying the outer electrical heating element 40. In at least some embodiments, the frame 33 has a significantly higher heat capacity than the membrane 32, and a higher heating power is applied to the frame 33 than to the membrane 32. In at least some embodiments, the electrical resistance element 6 is used to heat the frame 33 to a desired temperature.

In at least some embodiments, the electrical heating element 38 on the membrane 32 is electrically connected to contact fields 42 via the connecting conductor paths 39. In at least some embodiments, the first electrical resistance element 36 is electrically connected to contact fields 44. In at least some embodiments, the cantilevered wire 37 is electrically connected to the contact fields 46 via the connecting conductor path 41. In at least some embodiments, the outer electrical heating element 40 is electrically connected to contact fields 48. In at least some embodiments, the contact fields 42, 44, 46, 48 are made of the same material as the conductor paths forming the electrical resistance element 36, the cantilevered wire 37, the electrical heating element 38, the connecting conductor paths 39, 41 and the outer electrical heating element 40. In at least some embodiments, the contact fields 42, 44, 46, 48 are made of nickel. The contact fields 42, 44, 46, 48 are wider or have a larger diameter than the conductor paths connected to them. In at least some embodiments, all contact fields 42, 44, 46, 48 are arranged on one side of the frame 33 on the frame 33.

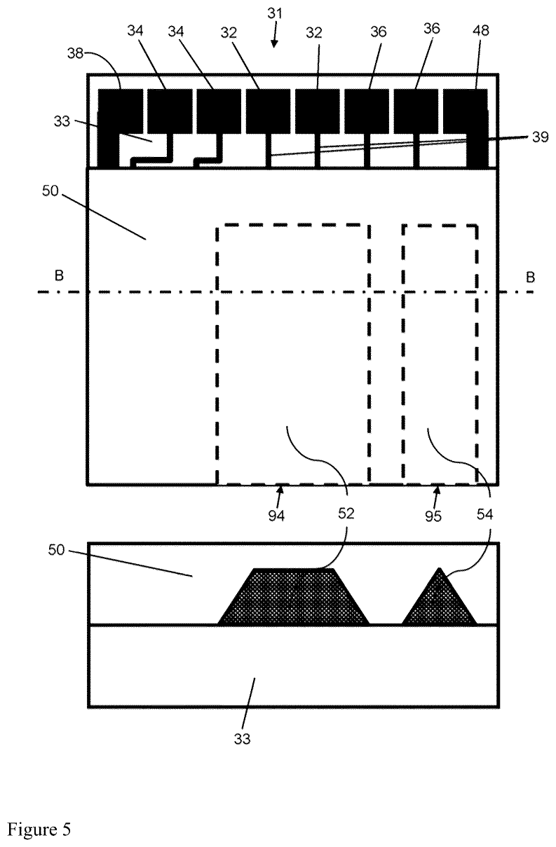

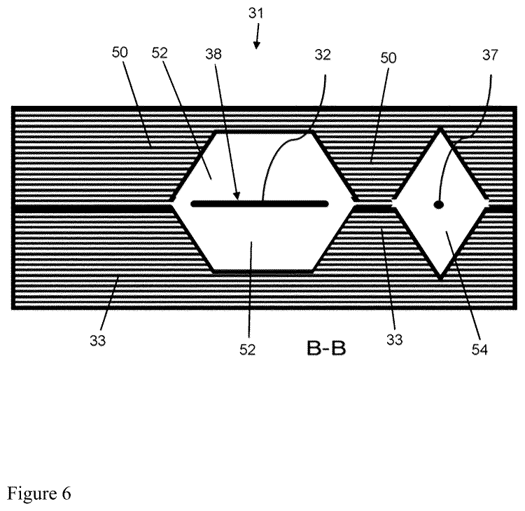

In at least some embodiments, the sensor element 31 shown in FIG. 4 includes one or more additional elements. The sensor element 31 also has a cover 50 which encloses a fluid volume 52 or a gas volume around the membrane 32. In at least some embodiments, the fluid volume 52 or gas volume forms the interior of a measuring chamber for the fluid medium or gas to be measured. In addition, the cover 50 encloses a fluid volume 54 or a gas volume around the cantilevered wire 37. In at least some embodiments, this fluid volume 54 or gas volume forms the interior of a reference measuring chamber. This is shown in FIGS. 5 and 6. The structure on the frame 33 and on the membrane 32 is identical to the sensor element 31 shown in FIG. 4. The cover 50 covers only the conductor paths, the membrane 32 and the upper side of the frame 33 shown in FIG. 4 except for the contact fields 42, 44, 46, 48 and adjacent regions, as shown in FIG. 5 above.

In at least some embodiments, the fluid volume 52 or gas volume around the membrane 32 is accessible via a gas inlet opening 94. In at least some embodiments, the conductor paths on the frame 33 are exposed in the region of the gas inlet opening 94 to the membrane 32. In at least some embodiments, the fluid volume 54 or gas volume around the cantilevered wire 37 is accessible via a gas inlet opening 95. In at least some embodiments, the conductor paths on the frame 33 are exposed in the region of the gas inlet opening 95 to the cantilevered wire 37.

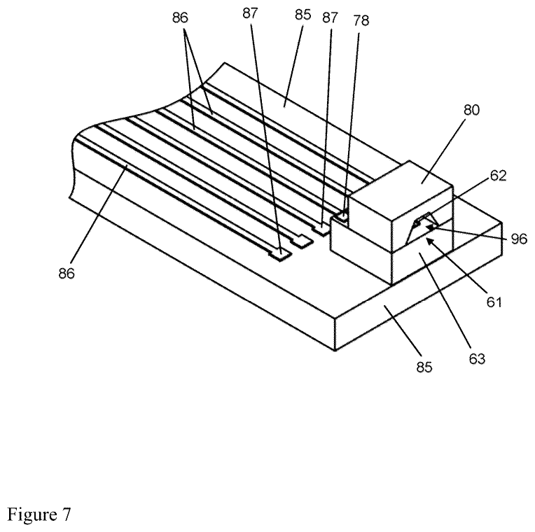

FIG. 7 shows a perspective view of a sensor element 61 according to at least one embodiment of the disclosure on a circuit board 85 for electrically contacting the sensor element 61. In at least some embodiments, the sensor element is constructed like the sensor element 1 according to FIGS. 1 to 3 or is constructed like the sensor element 31 according to FIGS. 4 to 6. Analogous to the first and second exemplary embodiments, the sensor element 61 has a cantilevered membrane 62 which is supported by a frame 63. In at least some embodiments, the sensor element 61 has contact fields 78 for electrically contacting an electrical heating element (not visible in FIGS. 7 to 9) on the membrane 62 and at least one electrical resistance element (not visible in FIGS. 7 to 9) on the frame 63. A cover 80 is provided to enclose a fluid volume 82 as a measuring chamber. In at least some embodiments, the fluid volume 82 is accessible via a gas inlet opening 96. In at least some embodiments, the conductor paths on the frame 63 are exposed in the region of the gas inlet opening 96.

On the circuit board 85, conductor paths 86 are arranged, which end in widened contact pads 87. In addition to the contact pads 87, the sensor element 61 are attached to the circuit board 85 in at least some embodiments. In at least some embodiments, each of the contact pads 87 is connected to one of the contact fields 78 by means of a bonding wire (not visible in FIG. 7).

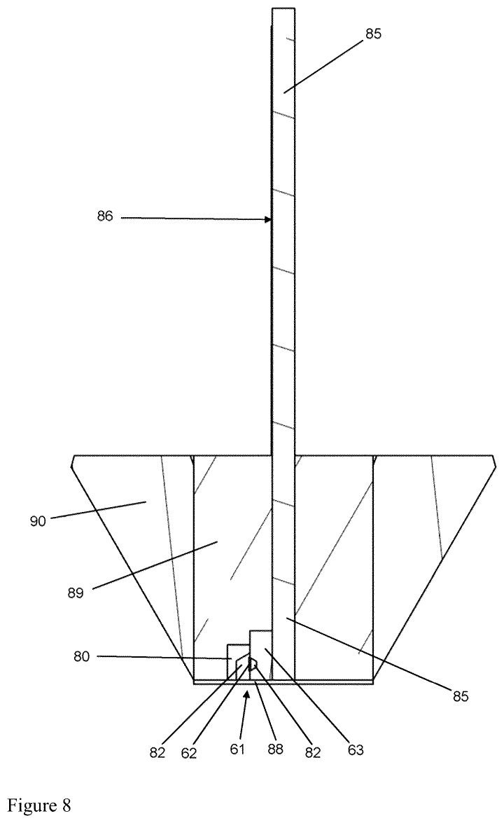

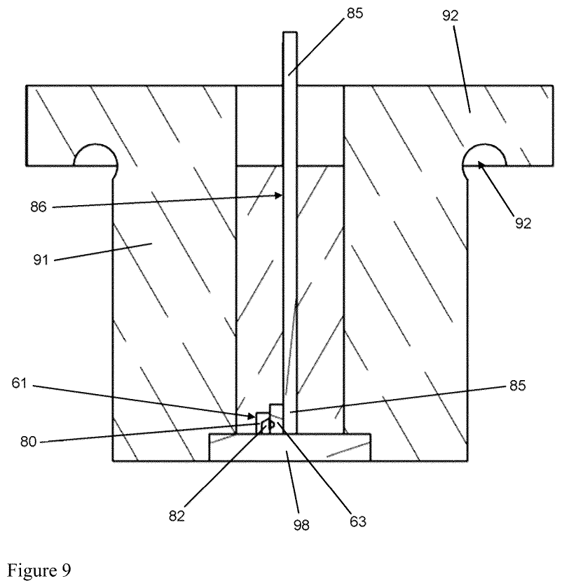

The arrangement of the contact pads 87 and the gas inlet opening 96 on opposite sides of the sensor provides a housing design that helps enable the contact pads 87 to be sealed or cast over without blocking the gas inlet opening. In at least some embodiments, this helps protect the contact pads against gas ingress, which is important in cases where corrosive gases could attack the contacts. This design is illustrated and described in detail in connection with FIGS. 8 and 9.

FIG. 8 shows a schematic cross-sectional view of a head of a sensor according to at least one embodiment of the disclosure, in which the sensor element 61 is installed on the circuit board 85 according to FIG. 7. The circuit board 85 is mounted in a housing 89 with an outer cone 90. In at least some embodiments, the housing 89 with the cone 90 is then installed in a suitable bore. In at least some embodiments, a filter 88 which is permeable to the gas to be measured is arranged on the front side of the sensor element 61. In at least some embodiments, the filter 88 is, for example, a sintered metal filter.

In at least some embodiments, the filter 88 is made of stainless steel or PTFE, or another material. In at least some embodiments, the gas to be measured passes by diffusion through the filter 88 into the measuring chamber of the sensor element 61. In at least some embodiments, this helps to avoid the measurement being distorted due to gas flow. At the same time, the filter 88 protects the sensor element from damage and contamination.

FIG. 9 shows a schematic cross-sectional view of another head of a further sensor according to at least one embodiment of the disclosure, in which the sensor element 61 is installed on the circuit board 85 according to FIG. 7. The circuit board 85 is mounted in a housing 91 with a circumferential groove 91. In at least some embodiments, the housing 91 is then installed in a suitable bore and sealed by an O-ring (not shown) arranged in the groove 91. In at least some embodiments, a filter 98 which is permeable to the gas to be measured is arranged on the front side of the sensor element 61. In at least some embodiments, the filter 98 is, for example, a sintered metal filter. In at least some embodiments, the filter 98 is made of stainless steel or PTFE, or another material. In at least some embodiments, the gas to be measured passes by diffusion through the filter 98 into the measuring chamber of the sensor element 61. In at least some embodiments, this helps to avoid the measurement being distorted due to gas flow. At the same time, the filter 98 protects the sensor element from damage and contamination.

In at least some embodiments, on the side of the circuit board 85 facing away from the sensor element 61, the sensor according to FIG. 8 and the sensor according to FIG. 9 are connected to at least one circuit for evaluation and at least one self-regulating Wheatstone bridge (not shown) for determining and regulating the heating voltage of the electrical heating element and thus the temperature of the electrical heating element on the membrane 62 as well as for determining and regulating the temperature of the frame 63 via the at least one electrical resistance element. These are connected to the respective conductor paths of the sensor element 61 via the conductor paths 86. In at least some embodiments, the at least one self-regulating Wheatstone bridge is arranged on a separate circuit board. In at least some embodiments, the self-regulating Wheatstone bridge is arranged on the sensor chip or on the sensor element 61. In at least some embodiments, other active elements, such as operational amplifiers, AD converters or interfaces, are assembled and/or implemented directly on the sensor chip or on the sensor element 61. In at least some embodiments, the sensor outputs a value for the thermal conductivity of the fluid medium as a signal.

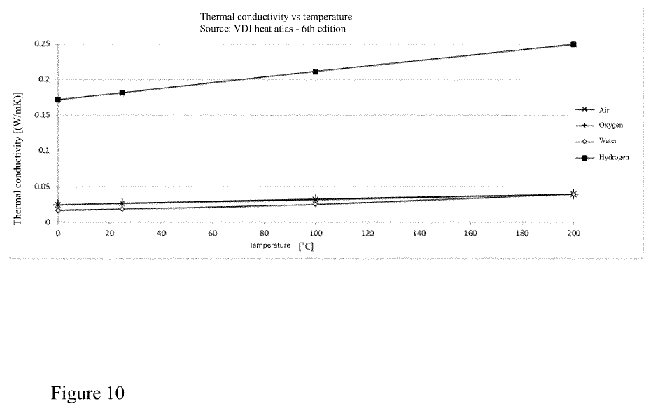

FIG. 10 shows the thermal conductivity in W/(m K) as a function of temperature in ° C. of gaseous hydrogen, water, air and oxygen. The thermal conductivity difference λ (T), e.g., at a temperature T of 100° C., between air and gaseous water is about 25 times smaller than between dry air and hydrogen. In at least some embodiments, this indicates that hydrogen can still be detected well in moist air, but measurement accuracy may be reduced. The thermal conductivity characteristic curves (as a function of temperature) of air and its components nitrogen and oxygen, as well as of gaseous water and hydrogen, all have a positive, but differently steep, slope. FIG. 10 shows how the thermal conductivities of air and gaseous water intersect at a temperature of approximately 200° C. and, together with hydrogen, form a quasi-binary gas mixture. In addition, the absolute thermal conductivity of hydrogen increases, which has a positive effect on the sensitivity of hydrogen measurement.

In at least some embodiments, the conversion of moist air with different H₂O concentrations into a quasi-binary gas mixture is achieved by the sensor or the sensor element 1, 31, 61 being set as near as reasonably possible to a target temperature in the range of 200° C. In at least some embodiments, both the temperature of the heat source on the membrane 2, 32, 62 and that of the heat sink (of the frame 3, 33, 63) are measured and controlled. In at least some embodiments, other fluctuating parameters such as pressure or flow rate of the fluid medium are compensated or kept constant. In at least some embodiments, the compensation is carried out using a reference measurement.

In at least some embodiments, the membrane 2, 32, 62, is a silicon nitride membrane (or an aluminum oxide membrane or a silicon oxide membrane) on which the electrical heating element 8, 38 is arranged, is a suspended membrane, and is largely free of mechanical stresses. In at least some embodiments, the membrane 2, 32, 62 is a silicon nitride membrane or a silicon oxide membrane. In at least some embodiments, the membrane 2, 32, 62 is a silicon nitride membrane. In at least some embodiments, the membrane 2, 32, 62 is a micro-electro-mechanical system (MEMS). In at least some embodiments, the membrane 2, 32, 62 is regulated as a heat source to a temperature of more than 200° C. In at least some embodiments, the temperature of the heat source (T_{q}) is set using an electrical circuit and calibration. By comparison, a general thermal conductivity MEMS with a continuous silicon oxide membrane is more susceptible to being destroyed by thermal stresses starting from 180° C.

The membrane area of the sensor elements according to at least one embodiment of the disclosure is kept as small as possible (for example, 0.3 by 0.3 mm²) in order to reduce the associated heating power and to increase the mechanical stability.

The distance d from the heat source on the membrane 2, 32, 62 to the heat sink (the frame 3, 33, 63) is determined by the gap provided by the recesses 4, 34 and an under-etching between the membrane 2, 32, 62 and the frame 3, 33, 63.

In at least some embodiments, at least two additional electrical resistance elements 6, 7, 36, and/or 37 are located on the frame 3, 33, 63. In at least some embodiments, the at least two additional electrical resistance elements are used to detect the temperature of the heat sink, or as a reference or compensation.

In at least some embodiments, including a reference resistor in the form of a suspended cantilevered wire 37 (FIGS. 4 to 6) helps enable a “real” reference. In at least some embodiments, this helps compensate for influences such as pressure or flow changes of the fluid medium or gas. In at least some embodiments, the reference has its own fluid channel 54 or gas channel, so that the heat does not reach from the heat source to the reference by convection or flow.

In at least some embodiments, the outer circumference of the frame 3, 33, 63 is surrounded by an electrical heating element 10, 40 that has a relatively low electrical resistance compared to the conductor paths 6, 7, 8, 36, 38. In at least some embodiments, this sets the temperature of the heat sink (Ts) of the entire frame 3, 33, 63 or chip to a desired value below 200° C.

In at least some embodiments, a cover 20, 50, 80 forms a bridge over the membrane 2, 32, 62 and protects the sensor element 1, 31, 61 or the sensor from mechanical damage as well as from dust and dirt. In at least some embodiments, the cover 20, 50, 80 is made of silicon. In at least some embodiments, this cover in turn defines the distance d above the membrane 2, 32, 62, which leads to a defined and reproducible measuring behavior of the sensor. On one side of the cover 20, 50, 80 there is a narrow channel for gas exchange by diffusion (see FIGS. 2 and 5 below).

In at least some embodiments, the chip design of the sensor element 1, 31, 61 includes features that make the thermal conductivity measurement robust and suitable for use in harsh and explosive environments. These include the following developments according to at least one embodiment of the disclosure:

All electrical contact fields 10, 12, 14, 16, 18, 40, 42, 44, 46, 48, 78 are located on the opposite side of the opening in the cover 20, 50, 80 for gas exchange. In at least some embodiments, the contact fields 10, 12, 14, 16, 18, 40, 42, 44, 46, 48, 78 are cast so that they are protected in corrosive environments.

The membrane 2, 32, 62 is in some cases sensitive to condensate contamination. The path to the membrane 2, 32, 62 therefore leads through a channel in the cover 20, 50, 80. In at least some embodiments, the channel opening is small, for example 0.3×0.25 mm² or less, and hinders access to the membrane 2, 32, 62 due to the surface tension between the liquid and the sensor walls. In at least some embodiments, a heating element 10, 40 or a section of the heating element 10, 40 with the temperature Ts is located transversely to this channel on the fixed frame 3, 33, 63. In at least some embodiments, this heating element 10, 40 or the section of the heating element 10, 40 causes most liquids, including water, to change into the gas phase and an overpressure is created in the small channel (measuring chamber), which further displaces the liquid from the channel opening.

The sensor design helps enable a small chip size (for example, about 2×2×1 mm³ or smaller) of the sensor element 1, 31, 61. In at least some embodiments, this and the integration of heating elements 6, 7, 8, 10, 36, 40 within the sensor element 1, 31, 61 helps to reduce the surface temperature as well as the heating power of the measuring system. These two parameters play an important role, in one or more embodiments, when used in potentially explosive atmospheres and when measuring explosive gases such as hydrogen gas in air or oxygen.

Further areas of application for sensor elements according to at least one embodiment of the disclosure include the measurement of helium-air-water gas mixtures for monitoring leaks and exploration, the measurement of hydrogen-carbon monoxide-water gas mixtures as synthesis gas, and the measurement of hydrocarbon-carbon monoxide-water gas mixtures for monitoring various methane reforming processes.

In a construction according to at least one embodiment of the disclosure, the contact fields 10, 12, 14, 16, 18, 40, 42, 44, 46, 48, 78 and the opening of the measuring chamber (see FIGS. 2 and 5 below), or the fluid volume 22, 52, 54, 82 or gas volume therein, are spatially separated from one another by the cover 20, 50, 80. In at least some embodiments, this helps to encapsulate the contact fields 10, 12, 14, 16, 18, 40, 42, 44, 46, 48, 78 with a durable material without closing the opening of the measuring chamber. In at least some embodiments, this design helps to close a gas channel flush when installing the sensor, or the sensor element 1, 31, 61, in a housing 89, 90, 91, and thus to significantly reduce the dead volume at the sensor. The flush connection helps enable the use of a filter 88, 98, for example, a sintered metal filter for protection against flow, explosion and dust without any gaps. The dead volume is the space between the sensor element and the distance to the surrounding components such as the filter 88, 98. In the construction according to at least one embodiment of the disclosure, there is little or no distance to the filter 88, 98 (see FIGS. 7 to 9). The dead volume is limited to the internal volume between the frame 3, 33, 63 and the support surface. The cover 20, 50, 80 is flush with the frame 3, 33, 63, so that together they form a flush surface.

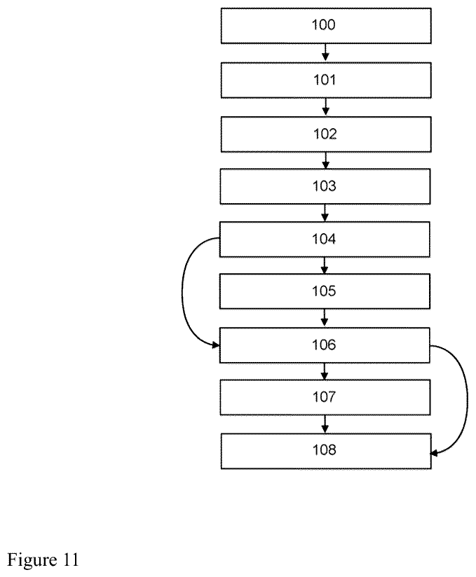

In the following, an exemplary method is explained using the sensor elements 1, 31 shown in FIGS. 1 to 6. The sequence of the exemplary method is shown schematically in FIG. 11.

In a first process step 100, the membrane 2, 32 is heated to a first temperature, to a temperature above 200° C., by means of the electrical resistance heating. At the same time, the frame 3, 33 is heated to a second temperature with the aid of the at least one electrical resistance element 6, 7, 36 and optionally with the electrical heating element 10, 40 on the frame 3, 33. In at least some embodiments, the second temperature is at least 1° C. lower than the first temperature. In at least some embodiments, the second temperature is below 200° C.

In a second process step 101, a fluid medium to be measured is fed into the fluid volume 22, 52 that surrounds the membrane 2, 32 on both sides. For this purpose, in at least some embodiments, the fluid medium first passes through a filter 88, 98. In at least some embodiments, the fluid medium is a gas mixture which, by diffusion through a sintered metal filter, reaches the gas volume surrounding the membrane 2, 32.

Furthermore, in a third process step 102, heat energy is transferred from the membrane 2, 32 and from the electrical heating element 8, 38 to the surrounding fluid medium.

In a fourth process step 103, the heat energy absorbed by the fluid medium is transferred to the frame 3, 33.

In a fifth process step 104, the temperature of the membrane 2, 32 is measured by determining the electrical resistance of the electrical heating element 8, 38 on the membrane 2, 32. At the same time, the temperature of the frame 3, 33 is determined by measuring the electrical resistance of the at least one electrical resistance element 6, 7, 36.

In an optional sixth process step 105, a reference measurement is carried out. In at least some embodiments, the temperature of the electrically heated cantilevered wire 37 is determined by the electrical voltage drop across the cantilevered wire 37, while the same fluid medium surrounds the cantilevered wire 37 and thereby transfers heat energy from the cantilevered wire 37 to the frame 33 by thermal conduction. In at least some embodiments, the cantilevered wire 37 is located in a fluid volume 54 separate from the fluid volume 52 surrounding the membrane 32.

In a seventh process step 106, the temperature of the membrane 2, 32 is controlled or regulated by regulating the electrical power for heating the electronic heating elements 8, 38 on the membrane 2, 32. In at least some embodiments, a self-regulating Wheatstone bridge circuit is used.

In an optional eighth process step 107, the temperature of the frame 3, 33 is controlled or regulated by regulating the electrical power for heating the at least one electrical resistance element 6, 7, 36 and/or the electrical heating element 10, 40 on the frame 3, 33. In at least some embodiments, a self-regulating Wheatstone bridge circuit is used.

In a subsequent ninth process step 108, the thermal property of the fluid medium, for example, the thermal conductivity and/or the hydrogen content, the hydrocarbon gas content or the helium content of the fluid medium, is determined by the electrical power required to regulate the temperature of the membrane 2, 32 and by the known temperature difference between the membrane 2, 32 and the frame 3, 33. In at least some embodiments, the optional sixth process step 105 has been carried out previously, and a correction is made when determining the thermal property of the fluid medium based on the reference measurement on the cantilevered wire 37. In at least some embodiments, this helps enable influences such as pressure changes or flow changes of the fluid medium to be measured and compensated.

The features of the disclosure disclosed in the foregoing description, as well as in the claims, figures and embodiments, may be essential both individually and in any combination for the realization of the disclosure in its various embodiments.

1. A sensor element for measuring thermal properties of a fluid medium the sensor element comprising: hydrocar
a membranes,
a cover;
a frame; and
at least one electrical resistance element that forms at least one conductor path on the frame and a first electrical heating element on the membrane that forms a conductor path on the membrane,
wherein:
the cover ends flush with the frame, so that the cover and the frame form a flush surface,
the frame surrounds the membrane on at least two sides,
the membrane is separated from the frame in regions by recesses, the membrane is cantilevered and connected to the frame by strips
the frame supports the membrane by the strips,
the first electrical heating element is electrically contacted by connecting conductor paths,
the connecting conductor paths run from the frame via at least one of the strips to the membrane,
the frame has a second electrical heating element that forms an outer conductor path on the frame, and
the second electrical heating element of the frame surrounds the at least one electrical resistance element on the frame on at least two sides.

2. The sensor element according to claim 1, wherein
the membrane is configured to be heated by the first electrical heating element and a temperature of the membrane is configured to be set by an electrical resistance of the first electrical heating element,
the sensor element has an adjustable heating device for heating the first electrical heating element and a membrane temperature setting device for setting the temperature of the membrane, and
the membrane temperature setting device is configured to determine the electrical resistance of the first electrical heating element.

3. The sensor element according to claim 1, wherein
a temperature of the frame is configured to be measured with the at least one electrical resistance element,
the sensor element has a frame temperature measuring device for measuring the temperature of the frame, and
the frame temperature measuring device is configured to determine an electrical resistance of the at least one electrical resistance element.

4. The sensor element according to claim 1, wherein the membrane and the frame are configured to be heated independently of one another.

5. The sensor element according to claim 1, wherein:
the strips and the membrane consist of a same material and are formed as a single piece.

6. The sensor element according to claim 1, wherein:
the frame consists of silicon or a ceramic.

7. The sensor element according to claim 1, wherein:
at least one of the following is satisfied:
the at least one electrical resistance element includes at least one metallic conductor path on the frame, and the at least one metallic conductor path consists of a metal or a metal alloy with a temperature coefficient of electrical resistance of at least 3×10⁻³ l/K or of nickel, and/or
the first electrical heating element by a metallic conductor path on the membrane, and the metallic conductor path consists of a metal or a metal alloy with a temperature coefficient of at least 3×10⁻³ l/K or of nickel.

8. The sensor element according to claim 1, wherein:
the membrane is not arranged in an edge region of the sensor element and is surrounded on all sides by the frame.

9. The sensor element according to claim 1, wherein:
the cover defines a gas volume around the membrane that is permeable to a surrounding gas.

10. The sensor element according to claim 1, wherein:
the sensor element has two electrical resistance elements that forms two conductor paths as the at least one electrical resistance element on the frame.

11. The sensor element according to claim 1, wherein:
the sensor element has a cantilevered wire as a reference resistor,
the cantilevered wire is connected at two ends to the frame and suspended from the frame and is spatially separated from the frame in some regions by recesses, and
the cover is spatially spaced from the cantilevered wire at least in some regions.

12. The sensor element according to claim 1, wherein:
the membrane is surrounded by the cover on at least two sides to the frame and above and below the membrane.

13. The sensor element according to claim 1, wherein
the outer conductor path of the second electrical heating element on the frame is wider than the conductor paths of the at least one electrical resistance element on the frame and of the first electrical heating element on the membrane.

14. The sensor element according to claim 13, wherein
an exposed part of the outer conductor path of the second electrical heating element of the frame runs in a fluid channel which leads to a fluid volume surrounding the membrane, and
the cover covers the other regions of the outer conductor path.

15. The sensor element according to claim 1, wherein:
the at least one electrical resistance element on the frame and the first electrical heating element on the membrane, are electrically contacted by contact fields on the frame.

16. The sensor element according to claim 1, wherein:
the membrane has an area of about 20 mm², or less.

17. The sensor element according to claim 1, wherein:
the frame has an outer diameter of about 10 mm or less.

18. The sensor element according to claim 1, wherein: the cover is heatable.

19. A sensor for determining thermal properties of a fluid medium, the sensor comprising:
the sensor element according to claim 1, wherein:
the membrane with the first electrical heating element on the membrane is heated independently of the frame, or the membrane with the first electrical heating element on the membrane and the frame are heated independently of each other,
a temperature of the membrane with the first electrical heating element on the membrane and a temperature of the frame are measured with the at least one electrical resistance element, and
the temperature of the membrane is regulated with the first electrical heating element on the membrane, and the temperature of the frame is regulated using the measured temperatures.

20. The sensor according to claim 19, wherein:
the sensor has a Wheatstone bridge circuit which is connected to the first electrical heating element on the membrane, and
heating power of the membrane is regulated using the Wheatstone bridge circuit.

21. The sensor according to claim 19, wherein
at least one of the following is satisfied:
the sensor is configured to heat the membrane to at least 200° C. during operation of the sensor, or
the sensor is configured to heat the frame to at least 130° C. during operation of the sensor.

22. A method for determining at least one thermal property of a fluid medium, the method comprising:
heating a membrane with a first electrical heating element that forms a conductor path arranged on the membrane,
the membrane is connected to a frame by strips and is held by the frame,
the membrane is surrounded by a defined volume of the fluid medium within a cover, and
the cover ends flush with the frame, so that the cover and the frame form a flush surface;
transferring heat from the membrane and the first electrical heating element to the fluid medium surrounding the membrane;
transferring heat from the fluid medium to the frame, wherein the frame has a lower temperature than the membrane;
determining temperatures of the membrane and the frame;
regulating the temperature of the membrane by regulating an electrical power for heating the membrane;
determining the thermal property of the fluid medium using by the electrical power that is used to regulate the temperature of the membrane and using a known temperature difference between the membrane and the frame; and
regulating the temperature of the frame.

23. The method according to claim 22, wherein
the regulating the temperature of the frame includes;
regulating the temperature of the frame by regulating an electrical power for heating the frame.

24. The method according to claim 22, wherein:
the temperature of the membrane is kept above 200° C., and
the temperature of the frame is kept below 200° C.

25. The method according to claim 22, wherein:
the membrane and the frame are part of a sensor,
the sensor has a known geometry and a known heating behavior, and
the sensor is calibrated prior to measurement in order to calculate, from the known geometry of the sensor and from the temperature difference between the membrane and the frame and the electrical power required to heat the membrane, the thermal property of the fluid medium.

26. (canceled)

27. The method according to claim 22, wherein:
the temperature of the membrane is regulated at least 5° C. and at most 200° C. above the temperature of the frame.

28. The method according to claim 22, wherein
the temperature of the membrane is maintained at at least 201° C. and at most 350° C.

29. The method according to claim 22, wherein:
the temperature of the frame is kept at least 135° C. and at most 199° C.

30. The method according to claim 22, wherein:
the temperature of the membrane is regulated in such a way, or the temperature of the membrane and the temperature of the frame are regulated such that;
an average value of the temperature of the frame and the temperature of the membrane is at least 180° C.

31. The method according to claim 22, wherein:
a temperature of a separate volume proximate to the frame is measured with a cantilevered wire, and
in the determining the thermal property of the fluid medium using the electrical power required to regulate the temperature of the membrane and using the temperature difference between the membrane and the frame:
the temperature measured with the cantilevered wire is used for correction in determining the thermal property of the fluid medium.

32. The method according to claim 22, wherein, in determining temperatures of the membrane and the frame,
the first electrical heating element is used.

33. The method according to claim 22, wherein:
the temperature of the membrane is kept constant using a first self-regulating Wheatstone bridge circuit, and
at least one of the following is satisfied:
the first electrical heating element is part of the first self-regulating Wheatstone bridge circuit, or
the temperature of the frame is kept constant using a second self-regulating Wheatstone bridge circuit or using a digital or computer-controlled controller.

34. The method according to claim 22, further comprising:
at least one of:
increasing or regulating the temperature of the fluid medium in a region proximate to a gas inlet to the membrane into the cover, or
increasing or regulating the temperature of the cover,
wherein the cover defines a gas volume around the membrane that is connected to a surrounding environment in a gas-permeable manner.

35. The method according to claim 22, wherein:
the frame is regulated to a temperature which is higher than a condensation temperature of a gas from the fluid medium.
